(19) United States
(12) Patent Application Publication
(10) Pub. No.: US 2026/0334857 A1
(43) Pub. Date: Oct. 29, 2026

(54) DENTAL DEVICE FOR DETECTING ORAL ACIDS AND METHODS REGARDING THE SAME

(71) Applicant: SOLVENTUM INTELLECTUAL PROPERTIES COMPANY, Maplewood, MN (US)

(72) Inventors: Yizhong WANG, Woodbury, MN (US); Ta-Hua YU, Woodbury, MN (US)

(21) Appl. No.: 19/113,277

(22) PCT Filed: Oct. 31, 2023
(86) PCT No.: PCT/IB2023/060978

§ 371 Date: Mar. 19, 2025

Related U.S. Application Data
(60) Provisional application No. 63/425,481, filed on Nov. 15, 2022.

(51) Int. Cl. A61B 5/00; A61C 7/08

(52) U.S. Cl. CPC A61B 5/4547; A61B 5/682; A61C 7/08; A61B 2560/0406

(57) ABSTRACT

The diagnostic dental device is for detecting the presence of microbes in an oral cavity. It includes a polymeric tray configured to receive one or more tooth of a subject. The dental device further includes an indicator composition disposed upon a tooth-contacting surface of the polymeric tray. The indicator composition includes a carrier polymer and an indicator compound that is used to detect the presence of microbial acids (such as lactic acid).

### BACKGROUND

Microbes in the oral environment are known to produce acids, such as lactic acid. It is further known that such acids dissolve tooth minerals and lead to white spot lesions. Continued exposure to microbial acids will lead to dental caries—a disease which requires invasive treatment to correct. While toothbrushing is generally considered to be an effective way to remove oral microbes at home, not everybody has the same technique, equipment, or tooth-cleaning compositions (e.g., toothpaste, mouthwash, or the like).

What is needed is a way to monitor microbial activity in the mouth. The present disclosure describes a dental device that allows a user to visually observe microbial activity by way of a color-changing indicator that is reactive to the presence of microbial acids.

### SUMMARY

In one embodiment, a diagnostic dental device is described. The dental device includes a polymeric tray configured to receive one or more tooth of a subject. The dental device further includes an indicator composition disposed upon a tooth-contacting surface of the polymeric tray. The indicator composition includes a carrier polymer and an indicator compound. The carrier polymer is characterized by a solubility in deionized water at 25° C. of less than 10 g/L and a solubility in 200 proof ethanol at 25° C. of greater than 1 g/L, and the carrier polymer excludes acidic moieties. The indicator compound is soluble in the carrier polymer at 25° C. of at least 0.01 g/L, and is represented by Formula I:

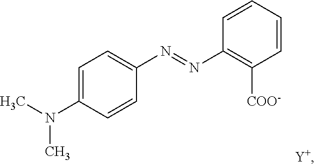

wherein Y⁺ is a pharmaceutically acceptable counterion.

In one embodiment, a method of detecting the presence acid on a tooth surface is described. The method includes providing a dental device described herein and inserting the dental device into a mouth of a subject such that one or more tooth surface is in contact with an indicator composition on the dental device. The method further includes determining whether or not a color change has occurred after a period of contact with the tooth surface.

In one embodiment, a method of recharging a dental device of the present disclosure is described. The method includes contacting the carrier polymer with an amount of a recharging solution. The recharging solution includes water and one or more of sodium bicarbonate, _potassium bicarbonate, sodium carbonate, potassium carbonate, sodium phosphate, potassium phosphate, and combinations thereof.

In one embodiment, a method of preparing a dental device of the present disclosure is described. The method includes providing a polymeric tray configured receive one or more teeth of a subject and applying an indicator composition to a tooth-contacting surface of the polymeric tray. The indicator composition includes a carrier polymer, an indicator compound of Formula I, and a solvent. The method further includes allowing the indicator composition to dry.

In one embodiment, a method of preparing a dental device of the present disclosure is described. The method includes providing a polymeric tray configured to receive one or more teeth of a subject, applying a carrier polymer solution to a tooth-contacting surface of the polymeric tray, and allowing the carrier polymer solution to dry to form a carrier polymer film. The carrier polymer solution includes a carrier polymer and a solvent. The method further includes applying an indicator solution to the carrier polymer film and allowing the indicator solution to dry. The indicator solution includes an indicator compound of Formula I and water.

In one embodiment, a kit is described. The kit includes a dental device of the present disclosure and a set of instructions directing a user insert the dental device into a mouth of a subject such that one or more tooth is in contact with the indicator composition.

### BRIEF DESCRIPTION OF THE DRAWINGS

FIG. 1 is a photograph of a clear tray aligner showing an activated indicator composition according to Example 3.

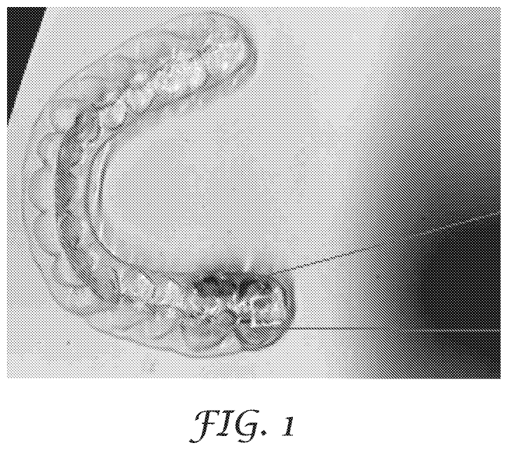

### DETAILED DESCRIPTION

Described herein are dental devices that allow users to monitor antimicrobial activity in an oral environment, which in many ways may indicate to the user the effectiveness of their oral hygiene routine. The dental devices include an indicator composition thereon, wherein an indicator compound within is characterized by one color at a pH above 4, and another color (e.g., red) at a pH below 4. Since oral microbes produce acid (e.g., lactic acid), a user may be able to visualize the presence of acid-producing bacteria. Moreover, the dental device of the present disclosure allows a user to determine specific areas within the oral environment that may host more microbes, e.g., red in some areas and not in others. In turn, this data may be used by the user to adjust their brushing technique, or the like, in order to prevent the development or progression of lesions and caries.

### Definitions

As used herein, “about” means±10 percent of a given value. For example, about 10 means 9 to 11.

As used herein, “acidic compounds” refer to compounds having an acidic functional group or moiety, such as carboxylic acid. The indicator compositions and/or carrier polymers exclude such groups and compounds given the nature of the intended indicator use. In other words, the presence of acidic groups may offset the pH of the environment and promote a color change that is not indicative of the presence of microbes, or is not indicative of a concerning amount of problematic microbes.

As used herein, “alkyl” means a linear or branched hydrocarbon chain. The hydrocarbon chain is saturated unless indicated otherwise. For example, a C₁₋₆ alkyl means a C₁ alkyl (i.e., methyl), a C₂ alkyl (i.e., ethyl), C₃ alkyl (i.e., propyl, isopropyl), C₄ alkyl (e.g., butyl, isobutyl, secbutyl), C₅ alkyl (e.g., pentyl), or C₆ (e.g., hexyl).

As used herein, “alkylenyl” refers to a bivalent alkyl group, i.e., —R—, where R includes ((CH)₂), and n may be defined by “C_{x-y}” or “Cₓ-C_{y}” where x and y are integers which indicate the number of carbon atoms. In some embodiments, alkylenyl groups may be optionally substituted, i.e., wherein one or more hydrogen is replaced with a defined group, e.g., —OH, C₁₋₄ alkyl, or the like.

As used herein, “aryl” describes a heteroatom-free aromatic moiety. An aromatic compound is cyclic, planar, fully conjugated, and follows Hückles Rule (i.e., having 4n+2 π-electrons, wherein n is an integer). For example, phenyl is a C₆ aryl, naphthyl and azulenyl are C₁₀ aryls, and anthracenyl and phenanthrenyl are C₁₄ aryls. An aryl group may be unsubstituted or substituted with groups such as C₁₋₆ alkyl optionally substituted with one or more halogens (e.g., —CF₃), C₁₋₆ alkoxy (e.g., —OCH₃, —OCH₂CH₃, or the like) optionally substituted with one or more halogens (e.g., —OCF₃), halogens (e.g., I, Br, Cl, F), or the like.

As used herein, “arylene” describes a bivalent aryl group, e.g., —R— where R is any aryl group described herein.

As used herein, “cycloalkylenyl” refers to a bivalent cycloalkyl group, e.g., cyclopropyl, cyclobutyl, cyclopentyl, cyclohexyl, or the like. Cycloalkylenyl is meant to include bicyclic and fused cycloalkyl groups where appropriate, e.g., isobornyl. The size of the any such cycloalkyl group is defined by “C_{x-y}” or “Cₓ-C_{y}” where x and y are integers which indicate the number of carbon atoms.

As used herein, “glass ionomer” refers to compositions that include a homopolymer or copolymer of an α,β unsaturated carboxylic acid, e.g., (meth)acrylate monomers described herein, and acid-substituted (meth)acrylate monomers described herein), a reactive glass filler such as fluoroaluminosilicate (FAS) and fluoroaluminoborate glass, and water. The homopolymer or copolymer may be referred to herein as a polycarboxylic acid or a polyacid.

As used herein, “(meth)acrylate” refers to a methacrylate compound or an acrylate compound, e.g., C(R)(R)═C(R)C(O)O—R, wherein each R is as described herein.

As used herein, the phrase “one or more of” such as used in the phrase “one or more of A and B” or “one or more of at least one A and at least one B” means a composition may include at least one A, more than one A, at least one B, more than one B, at least one A and at least one B, more than one A and more than one B. In other words, the phrase is not intended to mean the composition must have at least one of each of A and B.

As used herein, “optionally substituted” described a group that may or may not be substituted with a given substituent. The substitution reflects the exchange of a hydrogen for the given substituent. For example, an alkyl group, e.g., —CH₂CH₂CH₃ may or may not be (i.e., optionally) substituted with hydroxyl e.g., a group, —CH(OH)CH₂CH₃, —CH₂CH(OH)CH₃, —CH₂CH₂CH₂OH, or the like. The site of optional substitution is anywhere that includes an available hydrogen to be exchanged that would result in a stable compound.

As used herein, “polycarboxylic acid” or “polyacid” refer to a homopolymer or copolymer derived from monomers having an acidic moiety. For example, the homopolymers or copolymers derived from any combination of the (meth)acrylate or acid-substituted (meth)acrylate monomers would be considered a polycarboxylic acid or polyacid.

As used herein, “pharmaceutically acceptable” in terms of a “pharmaceutically acceptable salt” or a “pharmaceutically acceptable counterion” refers to salts or counterions that are well-established as being suitable for administration by any route to, or within, a human body. In other words, counterions that one skilled in art would deem safe to administer to a human. Thus, any counterion example is not to be construed to include forms (e.g., isotopes) that are not suitable for administration.

As used herein, “solubility” or “soluble” refers to the amount of precipitation of s given component in solution. Solubility at the stated concentration, under the stated conditions, may be determined by a skilled artisan by simply observing any phase separation of the indicator composition. One of ordinary skill in the art would be able to determine what components are soluble in other components, at the stated rates, without undue experimentation.

### Dental Devices

In various embodiments, a diagnostic dental device is described. The dental device may include a polymeric tray configured to receive one or more teeth of a subject, and an indicator composition disposed upon a tooth-contacting surface of the polymeric tray. The indicator composition may include a carrier polymer and an indicator compound. The carrier polymer may be characterized by a solubility in deionized water at 25° C. of less than 10 g/L and a solubility in 200 proof ethanol at 25° C. of greater than 1 g/L, and the carrier polymer may exclude acidic moieties. The indicator compound may be soluble in the carrier polymer at 25° C. of at least 0.01 g/L, and may be represented by Formula I:

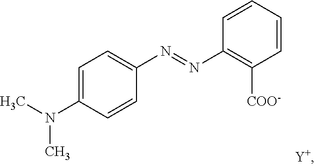

wherein Y⁺ may be a pharmaceutically acceptable counterion.

In some embodiments, the dental device may further include fluorides, such as amine fluoride, ammonium fluoride and quats with fluoride, these fluorides are organic solvent soluble to provide the coating compositions with no precipitation.

In some embodiments, the dental device may further include flavors to provide a pleasant mouth feel and breath freshening, these flavors can be mint, fruit, natural or artificial flavors.

Further details regarding the polymeric tray, the indicator composition, and the like are described below.

### Polymeric Tray

In many embodiments, the polymeric tray may include from any suitable polymeric material that is moldable to form a dental appliance, and once molded is generally conformable to a patient's teeth. The tray material may be transparent, translucent, or opaque. In some embodiments, the tray material is a clear or substantially transparent polymeric material that may include, for example, one or more of amorphous thermoplastic polymers, semi-crystalline thermoplastic polymers and transparent thermoplastic polymers chosen from polycarbonate, thermoplastic polyurethane, acrylic, polysulfone, polyprolylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone and combinations thereof. In another embodiment, the tray material may be chosen from clear or substantially transparent semi-crystalline thermoplastic, crystalline thermoplastics and composites, such as polyamide, polyethylene terephthalate. polybutylene terephthalate, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof. In some embodiments, the tray material may be chosen from polyethylene terephthalate, polyethylene terephthalate glycol, polycyclohexylenedimethylene terephthalate glycol, and mixtures and combinations thereof.

The polymeric tray may be made of a single polymeric material or may include multiple layers of the same or different polymeric materials. The material can be arranged in alternate layers such as, for example, in the arrangements ABA, BAB, BA, AB, etc. Exemplary suitable materials and configurations for polymeric tray may be found, for example, in U.S. Pat. No. 9,655,693 (Li et. al), U.S. Pat. No. 10,549,511 (Stewart et al.), as well as International Publication Nos. WO2020141440 (Liu et al.) and WO2020225657 (Yu et al.).

In many embodiments, the polymeric tray may be a dental aligner (i.e., clear tray aligner), a mouth guard, a retainer, or a night guard. The terms dental aligner, mouth guard, retainer, and night guard are well-known terms in the art. Dental aligners are commonly worn by users to realign teeth. Mouth guards are typically worn by users to prevent trauma to teeth during activities such as contact sports. Night guards are often worn by users to prevent grinding of teeth during sleep. Retainers are worn to arrest the teeth in a desired arrangement. Any of such devices may be used alongside the indicator compositions described herein to serve as a means for detecting the presence of acid on the surface of teeth. One may easily envision such devices, or similar devices, may be used for the methods described herein, whether they are marketed for, or are not marketed for, the above-intended use.

In other embodiments, the dental device is a bonded orthodontic appliance, such as an orthodontic bracket, band, or aligner attachment.

### Indicator Composition

In some embodiments, the indicator composition may be in the form of a film, a gel, a foam, a patterned coating, or a film applied through printing or imprinting process. In various embodiments, the indicator composition may form a continuous or a discontinuous array over a surface of the polymeric tray, or both. For example, some areas may be free of the indicator composition, while other areas have a dense arrangement of indicator composition. In another example embodiment, various areas may have indicator composition regions with varying shapes and feature spacings. The sizes and shapes vary widely, and need not be the same size or shape in a particular area of a polymeric tray surface, or over an entire surface.

In some embodiments, the indicator composition may include water in an amount of less than about 10% with respect to the weight of the indicator composition. For example, the indicator composition may include water in amount of less than 5 wt %, or within a range between any of the preceding values, e.g., between about 0.1 wt % and about 1 wt %, between about 1 wt % and about 5 wt %, or the like.

In some embodiments, the indicator composition may include a solvent in an amount of less than about 99.995 wt % with respect to the weight of the indicator composition. For example, the indicator composition may include a solvent in amount of less than 80 wt %, or a range between any of the preceding values, e.g., between about 80 wt % and about 90 wt %, between about 95 wt % and about 99.5 wt %, or the like.

In some embodiments, the carrier polymer may be present in the indicator composition in an amount of at least about 0.5 wt % with respect to the weight of the indicator composition. For example, the carrier polymer may be present in an amount in wt % with respect to the weight of the indicator composition of about 1%, or within a range between any of the preceding values, e.g., between about 2% and about 5%, between about 6% and about 10%, or the like.

In some embodiments, the indicator compound of Formula I may be present in an amount of at least 0.0001 wt % with respect to the weight of the indicator composition. For example, the indicator compound of Formula I may be present in an amount in wt % with respect to the weight of the indicator composition of about 0.005%, or within a range between any of the preceding values, e.g., between about 0.0075% and about 0.01%, between about 0.02% and about 0.1%, or the like.

### Carrier Polymer

In many embodiments, the carrier polymer may be characterized by a solubility in deionized water at 25° C. of less than about 100 g/L. For example, the carrier polymer may be characterized by a solubility in deionized water at 25° C., in g/L, of less than about 50 g/L, or within a range between any of the preceding values, e.g., between about 50 g/L and about 5 g/L, between about 4 g/and about 0.5 g/L, or the like. A low solubility in water is desirable to prevent solubilizing of the carrier polymer when a user rinses the dental device, and otherwise prevents the carrier polymer from dissolving within the mouth of a user. Further, a carrier polymer having a low solubility in water allows for the carrier polymer to remain on the dental device and be recharged (e.g., the indicator neutralized or a charge of indicator solution infused within the carrier polymer).

In many embodiments, the carrier polymer may be characterized by a solubility in 200 proof ethanol at 25° C. of greater than about 1 g/L. For example, the carrier polymer may be characterized by a solubility in 200 proof ethanol at 25° C., in g/L, of greater than about 5 g/L, or within a range between any of the preceding values, e.g., between about 5 g/L and about 10 g/L, between about 50 g/L and about 100 g/L, or the like. A high solubility in ethanol (or similar solvent) is desirable for preparing dental devices of the present disclosure, i.e., applying the carrier polymer and/or indicator composition solution to the polymeric tray. Likewise, a user has a simple means to remove and replace a carrier polymer from a dental device by simply dissolving the carrier polymer.

In some embodiments, the carrier polymer may be derived, at least in part, from one or more monomer represented by Formula II:

C(R¹)₂═C(R²)—X—C(O)—Z—R³, (II)
wherein:
each R¹ is independently selected from —H and a C₁₋₆ straight or branched alkyl optionally substituted with one or more of —OR⁴, —N(R⁴)₂, —OH, —SH, —NH₂, and —NHCO—,
R² is —H, or a C₁₋₆ straight or branched alkyl optionally substituted with one or more of —OR⁴, —N(R⁴)₂, —OH, —SH, —NH₂, and —NHCO—,
R³ is —H, or a C₁₋₆ straight or branched alkyl optionally substituted with one or more of —OR⁴, —N(R⁴)₂, —OH, —SH, —NH₂, and —NHCO—,
X and Z are independently selected from a bond, —O—, and —NR⁴—, and
each R⁴ is independently selected from —H and C₁₋₆ straight or branched alkyl, or
   two R⁴ are taken together with the atoms to which they are attached to form a 5- or 6-membered heterocycle,
wherein when Z is —O—, R³ is not —H.

In some embodiments, the carrier polymer may include one or more repeating units represented by Formula II′:

—[—C(R¹)₂—C(R²)(X—C(O)—Z—R³)—]—, (II′)
wherein:
   each R¹ is independently selected from —H and a C₁₋₆ straight or branched alkyl optionally substituted with one or more of —OR⁴, —N(R⁴)₂, —OH, —SH, —NH₂, and —NHCO—,
   R² is —H, or a C₁₋₆ straight or branched alkyl optionally substituted with one or more of —OR⁴, —N(R⁴)₂, and —OH, —SH, —NH₂, and —NHCO—,
   R³ is —H, or a C₁₋₆ straight or branched alkyl optionally substituted with one or more of —OR⁴, —N(R⁴)₂, and —OH, —SH, —NH₂, and —NHCO—,
   X and Z are independently selected from a bond, —O—, and —NR⁴—, and
   each R⁴ is independently selected from —H and C₁₋₆ straight or branched alkyl, or
      two R⁴ are taken together with the atoms to which they are attached to form a 5- or 6-membered heterocycle,
   wherein when Z is —O—, R³ is not —H.

In some embodiments, the carrier polymer may include one or more repeating units represented by Formula III′:

[—C(RA)2-C(RB)2-O—]—, (III′)
wherein:
each RA is —H, and
each RB is independently selected from —H and —CH3.

The carrier polymer may comprise at least 0.5 wt % units of Formula III′, with respect to the weight of the carrier polymer.

In the same or other embodiments, the carrier polymer comprises units represented by Formula IV′:

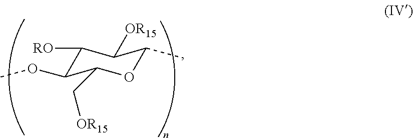

wherein:
   each R⁵ is independently selected from —H and —(CH₂)ₚ—OH, and
   n is an integer selected from 100, 500, 1000, 2000, 5000, 10000, and 100000, and
   p is an integer selected from 1, 2, and 3.

The carrier polymer may comprise at least about 0.1 wt %, and/or about 0.1 wt % to about 5 wt % units of Formula IV′, with respect to the weight of the carrier polymer.

In the same or other embodiments, the carrier polymer is derived, at least in part, from one or more vinyl dialkylamine monomer represented by Formula (V):

C(R⁶)₂═C(R⁷)—N(R⁸)(R⁹), (V)
wherein:
   each R⁶ is independently selected from —H and C₁₋₆ alkyl,
   R⁷ is —H or C₁₋₆ alkyl, and
   R⁸ and R⁹ are independently selected from a C₁₋₆ alkyl optionally substituted with one or more of —OR¹⁰, —N(R¹⁰)₂, and —OH, —SH, —NH₂, and —NHCO—, or
   R⁸ and R⁹, or R⁸ and R¹⁰, are taken together with the atoms to which they are attached to form a 5- or 6-membered heterocycle, the 5- or 6-membered heterocycle optionally substituted with oxo.

The carrier polymer may comprise at least about 0.1 wt %, and/or about 0.1 wt % to about 5 wt % units of Formula V, with respect to the weight of the carrier polymer.

In the same or other embodiments, the carrier polymer comprises units represented by Formula (V′):

—[—C(R6)2-C(R7)(N(R8)(R9))-]-, (V′)
wherein:
   each R6 is independently selected from —H and C1-6 alkyl,
   R7 is —H or C1-6 alkyl, and
   R8 and R9 are independently selected from a C1-6 alkyl optionally substituted with one or more of —OR10, —N(R10)2, and —OH, —SH, —NH2, and —NHCO—, or
   R8 and R9, or R8 and R10, are taken together with the atoms to which they are attached to form a 5- or 6-membered heterocycle, the 5- or 6-membered heterocycle optionally substituted with oxo.

The carrier polymer may comprise at least about 0.1 wt %, and/or about 0.1 wt % to about 5 wt % units of Formula V′, with respect to the weight of the carrier polymer.

In the same or other embodiments, the carrier polymer comprising units represented by Formula VI′:

—[C(R¹)₂—C(R²)(OH)]—]— (VI′)
wherein:
   each R¹ is independently selected from —H and a C₁₋₆ straight or branched alkyl optionally substituted with one or more of —OR⁴, —N(R⁴)₂, —OH, —SH, —NH₂, and —NHCO—, and
   R² is —H, or a C₁₋₆ straight or branched alkyl optionally substituted with one or more of —OR⁴,
   —N(R⁴)₂, —OH, —SH, —NH₂, and —NHCO—.

The carrier polymer may comprise at least about 0.1 wt %, and/or about 0.1 wt % to about 5 wt % units of Formula VI′, with respect to the weight of the carrier polymer.

### Indicator Compound

In many embodiments, the indicator compound of Formula I may be characterized by a solubility within the carrier polymer at 25° C. of at least 0.1 g/L. For example, the indicator compound of Formula I may be characterized by a solubility within the carrier polymer at 25° C., in g/L, of at least 0.001 g/L, or within a range between any of the preceding values, e.g., between about 0.001 g/L and about 0.01 g/L, between about 0.01 g/L and about 1 g/L, or the like. In other words, the selected carrier polymer must be able to dissolve at least 0.001 g/L of an indicator compound of Formula I in order to be a suitable carrier polymer. A carrier polymer that is incapable of hosting an indicator compound of Formula I may be an ineffective carrier polymer for allowing a user to determine site-specific acid production from local bacteria. Suitable carrier polymers are relatively more hydrophilic than those polymers incapable of hosting the indicator compound of Formula I. An indicator compound that is not soluble within the carrier polymer may not stay localized upon use of the dental device. In presently preferred embodiments, the indicator compound has certain interactions with the carrier polymer to reduce the extractable from the carrier polymer in solution, such interaction can be hydrogen bonding, acid/base interaction, and grafting to the polymer side chains.

The indicator compound of Formula I is a salt (i.e., an ionic compound consisting of a cation and an anion). In some embodiments, the indicator compound of Formula I has a counterion Y⁺ that may be monovalent, e.g., Li⁺ (lithium ion), Na⁺ (sodium ion), K⁺ (potassium ion), Cs⁺ (cesium ion), Ag⁺ (silver ion), or R₄N⁺ (quaternary ammonium ion). In other embodiments, the counterion may be divalent, e.g., Ca²⁺ (calcium ion) or Mg²⁺ (magnesium ion), along with a suitable anion such as 2-[[4-(dimethylamino)phenyl]diazenyl]benzoate, Cl⁻ (chloride ion), or the like. In some embodiments, the counterion may be Na⁺.

### Methods of Use

In various embodiments, a method of detecting the presence acid on a tooth surface is described. The method may include providing a dental device described herein and inserting the dental device into a mouth of a subject such that one or more tooth surface is in contact with an indicator composition on the dental device. The method may further include determining whether or not a color change has occurred after a period of contact with the tooth surface. In some embodiments, the method may further include determining whether or not a color change has occurred within 60 minutes or less after consuming food or drink.

### Methods of Preparing

In various embodiments, a method of preparing a dental device of the present disclosure is described. The method may include providing a polymeric tray configured receive one or more tooth of a subject and applying an indicator composition to a tooth-contacting surface of the polymeric tray. The indicator composition may include a carrier polymer, an indicator compound of Formula I, and a solvent. The method may further include allowing the indicator composition to dry.

### Step-Wise Preparation

In various embodiments, a method of preparing a dental device of the present disclosure is described. The method may include providing a polymeric tray configured to receive one or more tooth of a subject, applying a carrier polymer solution to a tooth-contacting surface of the polymeric tray, and allowing the carrier polymer solution to dry to form a carrier polymer film. The carrier polymer solution may include a carrier polymer and a solvent. The method may further include applying an indicator solution to the carrier polymer film and allowing the indicator solution to dry. The indicator solution may include an indicator compound of Formula I and water.

The carrier polymer solution and/or indicator solution can be applied on the surfaces by any suitable printing or deposition technique. In some non-limiting examples, screen printing is a printing technique in which a mesh is used to transfer a liquid composition, referred to as an ink, onto a substrate, except in areas made impermeable to the ink by a blocking stencil. A blade or squeegee is moved across the screen to fill the open mesh apertures with ink, and a reverse stroke then causes the screen to touch the substrate momentarily along a line of contact. This causes the ink to wet the substrate and be pulled out of the mesh apertures as the screen springs back after the blade has passed. One color is printed at a time, so several screens can be used to produce a multi-colored image or design.

In another non-limiting example, a flexographic print is made by creating a positive mirrored master of the required image as a 3D relief in a rubber or polymeric material, which is referred to as a flexographic printing plate. The image areas on the flexographic printing plate are raised above the non-image areas on the plate. Printing ink is transferred to the image areas of the flexographic plate via an anilox roll (composed of cells filled with ink, usually by a blading ink into the anilox roll cells), and then the ink is transferred to the substrate by contacting the “inked” flexographic plate to the substrate. Ink is only transferred from the relief features of the flexographic plate to the substrate.

In another embodiment, the composition may be indirectly applied to surfaces of the dental device. For instance, a carrier and/or indicator compositions can be applied to a transfer substrate by at least one suitable printing process as described in detail above to form release structures. The transfer substrate is contacted with a polymeric material forming (or used in forming) the dental device such that the release structures contact and transfer to a major surface thereof. This lamination step may optionally include at least one of heating or pressure to facilitate the transfer. The transfer substrate is then peeled away so that the portions of the transfer layer not overlain by the release structures transfer to the surface of the substrate. Exemplary methods of deposition and transfer are set forth in International Publication Nos. WO2021137159 (Velamakanni et al.) and WO2022162488 (Wang et al.), as well as the Examples below.

### Methods of Recharging

In various embodiments, a method of recharging a dental device of the present disclosure is described. The method may include contacting the carrier polymer with an amount of a recharging solution. The recharging solution may include water and one or more of sodium bicarbonate, potassium bicarbonate, sodium carbonate, potassium carbonate, sodium phosphate, potassium phosphate, and their mixtures.

A recharging solution can be added to the tray to reverse the change in color, allowing continued use of the tray after the indicator compound has provide a warning on acid production from local bacteria. if the tray changed the color due to the interaction of the microbial produced acid and the indicator, it lost the indication function. Since the tray will be used by the patients for many days, it is necessary to reverse the color change and recharging the indicator for the future color change indication. These reverse or recharging of the coating can be achieved by soaking the tray with some chemical solutions or reapply the indication coating. Further details of example recharging solutions of the present disclosure are described in more detail below.

In some embodiments, the method may further include contacting the carrier polymer with an indicator solution. The indicator solution may include an indicator compound of Formula I and water. Further details of indicator solutions of the present disclosure are described in more detail below.

In some embodiments, the method may further include removing the carrier polymer from the polymeric tray and applying a fresh indicator composition. The removing may include contacting the carrier polymer with a solvent. The solvent may be selected from, for example, a C₂₋₃ alcohol (e.g., ethanol, isopropanol) acetone, or combinations thereof. Applying a fresh indicator composition may include applying a carrier polymer solution described in detail below, and then applying an indicator solution described in detail below. Alternatively, applying a fresh indicator composition may include applying an indicator composition solution including a carrier polymer, an indicator compound, and one or more solvents as listed above, described in further detail below.

### Kits

In various embodiments, a kit is described. The kit may include a dental device of the present disclosure and a set of instructions directing a user insert the dental device into a mouth of a subject such that one or more tooth is in contact with the indicator composition.

In some embodiments, the kit may further include one or more of: a recharging solution, an indicator solution, a carrier polymer solution, and an indicator composition solution. Details for each of the aforementioned solutions are described in further detail below.

In some embodiments, the kit may further include a recharging solution.

In some embodiments, the kit may further include a recharging solution and an indicator solution.

In some embodiments, the kit may further include a recharging solution and an indicator composition solution.

In some embodiments, the kit may further include an indicator solution.

In some embodiments, the kit may further include an indicator solution and a carrier polymer solution.

In some embodiment, the kit may further include an indicator composition solution.

Kits of the present disclosure may also include one or more appliances in addition to the indicator and carrier polymer solutions.

### Recharging Solutions

In some embodiments, the kit may further include a container having a recharging solution therein. The recharging solution may include sodium bicarbonate, potassium bicarbonate, sodium carbonate, potassium carbonate, sodium phosphate, potassium phosphate, or a combination thereof.

In some embodiments, the recharging solution may be an aqueous solution of sodium bicarbonate. Sodium bicarbonate may be present in an amount of about 0.5 wt % to about 5 wt %.

In some embodiments, the recharging solution may consist essentially of water and sodium bicarbonate.

### Indicator Solutions

In some embodiments, the kit may further include a container having an indicator solution therein. The indicator solution may include an indicator compound of Formula I and water.

In some embodiments, the indicator solution may include an indicator compound of I present in an amount of about 0.0001 wt % to about 0.1 wt %, based on the total weight of solution.

In some embodiments, the indicator solution may further include a supplemental indicator compound of Formula VII. In some embodiments, the indicator solution may include a supplemental indicator compound of Formula VII present in an amount of about 0.001 wt % to about 0.1 wt %. In some embodiments, the indicator solution may include an indicator compound of Formula I present in an amount of about 0.005 wt % to about 0.01 wt %, and a supplemental indicator compound of Formula VII present in an amount of about 0.001 wt % to about 0.1 wt %. In some embodiments, the indicator solution may include an indicator compound of Formula I and a supplemental indicator compound of Formula VII present in a molar ratio of about 1:1.

In other embodiments, the indicator solution may consist essentially of an indicator compound of Formula I and water.

In other embodiments, the indicator solution may consist essentially of an indicator compound of Formula I, water and a supplemental indicator compound of Formula VII:

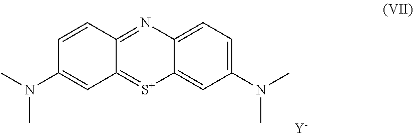

wherein: Y⁻ is a pharmaceutically acceptable counterion.

The supplement indicator compound of Formula VII may be present in an amount of about 0.001 wt % to about 0.05 wt %, with respect to the weight of the indicator composition. Moreover, the indicator compound of Formula I and the supplement indicator compound of Formula VII may be present in a molar ratio of about 1:2.5.

In many embodiments, indicator solution excludes any solvent in which the carrier polymer is soluble within in an amount of at least 0.01 g/L at 25° C. It is not desirable to apply a solution having a solvent according to the above since said solvent may dissolve the carrier polymer on a polymeric tray.

### Carrier Polymer Solutions

In some embodiments, the kit may further include a container having a carrier polymer solution therein. The carrier polymer solution may include any carrier polymer described herein and a solvent. Suitable solvents include any solvent in which the carrier polymer is soluble within in amount of at least 1 g/L at 25° C. Suitable solvents may be further characterized by a boiling point of less than 100° C. For example, the carrier polymer solution may include a solvent selected from a C₂₋₃ alcohol (e.g., ethanol, isopropanol), acetone, and a combination thereof.

In some embodiments, the carrier polymer may be present in the carrier polymer solution in an amount of about 0.1 wt % to about 10 wt % with respect to the weight of the carrier polymer solution.

In some embodiments, the solvent may be present in the carrier polymer solution in amount of about 80 wt % to about 99.95 wt % with respect to the weight of the carrier polymer solution.

In some embodiments, the carrier polymer solution may consist essentially of a carrier polymer and a C₂₋₃ alcohol solvent. In some embodiments, the carrier polymer may be present in an amount of about 0.1 wt % to about 10 wt % with respect to the weight of a carrier polymer solution consisting essentially of the carrier polymer and a C₂₋₃ alcohol.

In some embodiments, the carrier polymer solution may consist essentially of hydrophilic acrylate(s).

### Indicator Composition Solutions

In some embodiments, the kit may further include a container having an indicator composition solution therein. The indicator composition solution may include any carrier polymer described herein, an indicator compound of Formula I, and a solvent. Suitable solvents include any solvent in which the carrier polymer is soluble within in amount of at least 0.1 g/L at 25° C. Suitable solvents may be further characterized by a boiling point of less than 100° C.

In some embodiments, the carrier polymer may be present in the indicator composition solution in an amount of about 0.1 wt % to about 10 wt % with respect to the weight of the indicator composition solution.

In some embodiments, the indicator compound of Formula I may be present in the indicator composition solution in an amount of about 0.0001 wt % to about 0.1 wt % with respect to the weight of the indicator composition solution.

In some embodiments, the solvent may be present in the indicator composition solution in amount of about 60 wt % to about 99.9 wt % with respect to the weight of the indicator composition solution.

In some embodiments, the indicator composition may further include a co-indicator.

In some embodiments, the indicator composition solution may consist essentially of a carrier polymer, an indicator compound of Formula I, and a C₂₋₃ alcohol solvent. In some embodiments, the carrier polymer may be present in an amount of about 0.5 wt % to about 10 wt %, and the indicator compound of Formula I may be present in an amount of about 0.001 wt % to about 0.5 wt % with respect to the weight of an indicator composition solution consisting essentially of the carrier polymer, the indicator compound of Formula I, and the C₂₋₃ alcohol.

In some embodiments, the indicator composition solution may consist essentially of hydrophilic acrylate(s).

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

TABLE 1
Materials List

| ABBREVIATION/ | | |
|---|---|---|
| NAME | DESCRIPTION | SOURCE |
| E100 | EUDRAGIT E100 | Evonik |
| | Basic cationic copolymer based on | Industries |
| | dimethyl aminoethyl methacrylate, | |
| | butyl methacrylate, and methyl | |
| | methacrylate | |
| L100 | EUDRAGIT L100 | Evonik |
| | Acidic copolymer based on methacrylic | Industries |
| | acid and methyl methacrylate | |
| PMMA | Poly (methyl methacrylate) | Sigma |
| | | Aldrich |
| CH₂Cl₂ | Dichloromethane or methylene chloride | Alfa Aesar |
| IPA | Isopropyl alcohol | Alfa Aesar |
| Methyl red | Methyl red sodium salt (CAS # 845-10- | Alfa Aesar |
| | 3) | |
| Na₂HPO₄—12H₂O | Sodium phosphate dibasic | Alfa Aesar |
| | dodecahydrate | |
| CaCl₂ dihydrate | Calcium chloride dihydrate | Alfa Aesar |
| HEC 250HHX | hydroxyethyl cellulose; HEC250 pharm | Ashland |
| pharm | M grade | |
| Lactic acid | 90% lactic acid | Alfa Aesar |

### Preparatory Example Coating Compositions

### PEx.1—Organic Solvent-Based Coating Composition

A solvent based coating composition, Preparatory Example PEx.1, was prepared according to Table 2. The listed components were combined and mixed well to form a low viscosity solution with slight yellow color. Alternatively, ethanol or another solvent may be used to dissolve the EUDRAGIT E100 basic acrylate copolymer. The polymer solution needs to be neutral to slightly basic pH.

TABLE 2
Preparatory Example PEx. 1, Solvent-Based Coating Composition

| Chemical | Percent % |
|---|---|
| IPA | 97.99 |
| E100 polymer | 2.0 |
| methyl red | 0.01 |
| total | 100.0 |

### PEx.2-PEx.4—Aqueous-Based Coating Compositions

Aqueous-based coating compositions of Preparatory Examples PEx.2, PEx.3, and PEx.4, were prepared according to Table 3. The listed components were combined and mixed well to form a gel type solution with yellow color.

TABLE 3
Preparatory Examples PEx. 2, Aqueous-
Based Coating Composition

| | PEx. 2 | PEx. 3 | PEx. 4 |
|---|---|---|---|
| Chemical | Percent (%) | Percent (%) | Percent (%) |
| water | 98.89 | 98.87 | 98.877 |
| HEC 250HHX pharm | 1.0 | 1.0 | 1.0 |
| Na₂HPO₄—12H₂O | 0.1 | 0.1 | 0.1 |
| methyl red | 0.01 | 0.01 | 0.0025 |
| CaCl₂ dihydrate | — | 0.02 | 0.02 |
| Total | 100.0 | 100.0 | 100.0 |

### Comparative Preparatory Example Coating Compositions

Comparative preparatory example coating compositions CPEx.1-CPEx.3 were prepared according to components listed in Table 4.

TABLE 4
Comparative Preparatory Examples CPEx. 1-CPEx. 3

| | CPEx. 1 | CPEx. 2 | CPEx. 3 |
|---|---|---|---|
| Chemical | Percent (%) | Percent (%) | Percent (%) |
| IPA | 97.99 | — | 97.99 |
| CH₂Cl₂ | — | 97.99 | — |
| PMMA | 2.0 | 2.0 | — |
| L100 | — | — | 2.0 |
| Methyl | 0.01 | 0.01 | 0.01 |
| red | | | |
| Total | 100.0 | 100.0 | 100.0 |
| Results: | PMMA did | PMMA based | Acidic polymer-based |
| | not dissolve | coating failed to | coating L100 caused |
| | in IPA | change color when | the indicator to |
| | | exposed to lactic | change to red color |
| | | acid. | during coating |
| | | | preparation. |

### Color Change Testing of Example Compositions Coated on Clear Tray Aligners

Example aligners Ex.1-Ex.4 were prepared in the following manner. Each of the prepared Preparatory Example Coating Compositions, PEx.1-PEx.4, described above were spot coated as a thin layer onto the inside of an orthodontic aligner at the molar region and allowed to dry. The orthodontic aligner used was commercially available 3M CLARITY ALIGNER FLEX, available from 3M Company of St. Paul, MN, USA. A 2% solution of lactic acid was prepared by diluting a commercially available 90% lactic acid solution with water. One drop of the 2% lactic acid solution was dropped onto the dried spot of Preparatory Example Composition that had been dried onto inside the molar region of each test aligner. For each of the Example Compositions the dried spot turned red, indicating the exposure to acid. An additional example Ex.4T was prepared in the same fashion as Example Ex.4, however the drop of 2% lactic acid solution was placed on the occlusal/lingual portion of the back molar of a typodont model. This was to simulate lactic acid from microbes present on a molar in an oral environment, which can lead to caries. This aligner had been thermoformed to fit the lactic acid treated typodont model. The aligner that had been spot treated with PEx.4 in the corresponding molar region was placed onto the typodont model with treated with lactic acid. The portion of the aligner Ex.4T that had been spot treated with PEx.4 turned red when exposed to the lactic acid that had been placed on the typodont model.

TABLE 5
Examples Ex. 1-Ex. 4B, Aligners with
Color changing Indicator coating

| Example Aligners: | Ex. 1 | Ex. 2 | Ex. 3 | Ex. 4 | Ex. 4T |
|---|---|---|---|---|---|
| Preparatory Example | PEx. | PEx. | PEx. | PEx. | PEx. |
| Coating Composition: | 1 | 2 | 3 | 4 | 4 |
| Dried spot color on aligner | clear | pale | pale | pale | pale |
| before lactic acid | | yellow | yellow | yellow | yellow |
| Color of Example spot after | red | red | red | red | red |
| exposed to one drop 2% | | | | | |
| lactic acid | | | | | |

The patents, patent documents, and patent applications cited herein are incorporated by reference in their entirety as if each were individually incorporated by reference. Although specific embodiments of the present disclosure have been shown and described herein, it is understood that these embodiments are merely illustrative of the many possible specific arrangements that can be devised in application of the principles of the present disclosure. Numerous and varied other arrangements can be devised in accordance with these principles by those of ordinary skill in the art without departing from the spirit and scope of the present disclosure. Thus, the scope of the present disclosure should not be limited to the structures described in this application, but only by the structures described by the language of the claims and the equivalents of those structures.

1. A diagnostic dental device comprising:
a polymeric tray configured to receive one or more tooth of a subject, and
an indicator composition disposed upon on a tooth-contacting surface of the polymeric tray,
the indicator composition comprising:
a carrier polymer characterized by:
a solubility in deionized water at 25° C. of less than 10 g/L,
a solubility in 200 proof ethanol at 25° C. of greater than 1 g/L, and excludes acidic moieties;
an indicator compound represented by Formula I: 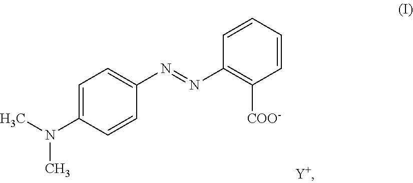
wherein Y⁺ is a pharmaceutically acceptable counterion,
wherein the indicator compound is soluble in the carrier polymer at 25° C. of at least 0.001 g/L.

2. The diagnostic dental device of claim 1, the polymeric tray comprising a polyurethane, copolyester ether, copolymers of ethylene and (meth)acrylates, polyethylene terephthalate (PET), polyethylene terephthalate glycol (PETg), polycyclohexylenedimethylene terephthalate (PCT), 2,2,4,4-tetramethyl-1,3-cyclobutanediol modified poly cyclohexylenedimethylene terephthalate, PEN, and mixtures and combinations thereof.

3. The diagnostic dental device of claim 1, the polymeric tray being a dental aligner, a mouth guard, a retainer, or a night guard.

4. The diagnostic dental device of claim 1, wherein the indicator composition is a film.

5. The diagnostic dental device of claim 1 wherein the carrier polymer derived, at least in part, from one or more acrylate monomer, one or more acrylamide monomer, one or more alkylene oxide, one or more modified cellulose oligomer, one or more vinyl dialkylamine, or a combination thereof.

6. The diagnostic dental device of claim 1, the carrier polymer characterized by the presence of a nitrogen atom in a functional group.

7. The diagnostic dental device of claim 1, the carrier polymer derived, at least in part, from one or more monomer represented by Formula II:
C(R¹)₂═C(R²)—X—C(O)—Z—R³, (II)
wherein:
each R¹ is independently selected from —H, -Ph, and a C₁₋₆ straight or branched C₁₋₆ alkyl optionally substituted with one or more of —OR⁴, —N(R⁴)₂, and —OH, —SH, —NH₂, and —NHCO—,
R² is —H, or a C₁₋₆ straight or branched alkyl optionally substituted with one or more of —OR⁴, —N(R⁴)₂, and —OH, —SH, —NH₂, and —NHCO—,
R³ is R⁵, —H, or a C₁₋₆ straight or branched C₁₋₆ alkyl optionally substituted with one or more of —OR⁴OR⁵, —N(R⁴R⁵)₂, and —OH, —SH, —NH₂, and —NHCO—,
X and Z are independently selected from a bond, —O—, and —NR⁴NR⁵—, and
each R⁴ is independently selected from —H and C₁₋₆ straight or branched C₁₋₆ alkyl, and
each R⁵ is independently selected from —H and straight or branched C₁₋₆ alkyl, or
two R⁴ R⁵ are taken together with the atoms to which they are attached to form a 5- or 6-membered heterocycle,
wherein when Z is —O—, R³ is not —H.

8. The diagnostic dental device of claim 7, the carrier polymer derived from a monomer of Formula II,
wherein:
X is a bond and Z is —O—, or
X is —O— and Z is a bond.

9. The diagnostic dental device of claim 1, the carrier polymer derived from a monomer represented by Formula II-a:
C(R¹)₂═C(R²)—C(O)—O—R³, (II-a)
wherein:
each R¹ is —H,
R² is —H or —CH₃,
R³ is a C₂₋₃ alkyl substituted with —OR⁴ or —N(R⁴R⁵)₂, and
R⁴ is —H or C₁₋₆ alkyl.

10. The diagnostic dental device of claim 7, the carrier polymer derived from a monomer of Formula II,
wherein:
X is a bond and Z is —NR⁴NR⁵—, or
X is —NR⁴NR⁵— and Z is a bond.

11. The diagnostic dental device of claim 1, the carrier polymer derived from a monomer represented by Formula II-b:
C(R¹)₂═C(R²)—C(O)—N(R⁴R⁵)—R³, (II-b)
wherein:
each R¹ is —H,
R² is —H or —CH₃, and
R³ and R⁴ are —H.

12. The diagnostic dental device of claim 1, the carrier polymer comprising units represented by Formula II′:
—[—C(R¹)₂—C(R²)(X—C(O)—Z—R³)—]—, (II′)
wherein:
each R¹ is independently selected from —H and a C₁₋₆ straight or branched alkyl optionally substituted with one or more of —OR⁴, —N(R⁴)₂, —OH, —SH, —NH₂, and —NHCO—,
R² is —H, or a C₁₋₆ straight or branched alkyl optionally substituted with one or more of —OR⁴, —N(R⁴)₂, and —OH, —SH, —NH₂, and —NHCO—,
R³ is R⁵, —H, or a C₁₋₆ straight or branched C₁₋₆ alkyl optionally substituted with one or more of —OR⁴OR⁵, —N(R⁴R⁵)₂, and —OH, —SH, —NH₂, and —NHCO—,
X and Z are independently selected from a bond, —O—, and —NR⁴NR⁵—, and
each R⁴ is independently selected from —H and C₁₋₆ straight or branched C₁₋₆ alkyl, and
each R⁵ is independently selected from —H and straight or branched C₁₋₆ alkyl, or
two R⁴ R⁵ are taken together with the atoms to which they are attached to form a 5- or 6-membered heterocycle,
wherein when Z is —O—, R³ is not —H.

13. The diagnostic dental device of claim 12, carrier polymer comprising least 5 wt % units of Formula II′, with respect to the weight of the carrier polymer.

14. The diagnostic dental device of claim 13, the carrier polymer comprising about 5 wt % to about 35 wt % units of Formula II′, with respect to the weight of the carrier polymer.

15. The diagnostic dental device of claim 1, wherein the carrier polymer is derived, at least in part, from one or more alkylene oxide selected from ethylene oxide and propylene oxide.

16. The diagnostic dental device of claim 1, the carrier polymer comprising units represented by Formula III′:
—[—C(R^{A})₂—C(R^{B})₂—O—]—, (III′)
wherein:
each R^{A} is —H, and
each R^{B} is independently selected from —H and —CH₃.

17. The diagnostic dental device of claim 16, the carrier polymer comprising at least 0.5 wt % units of Formula III′, with respect to the weight of the carrier polymer.

18. The diagnostic dental device of claim 16, the carrier polymer comprising about 0.1 wt % to about 5 wt % units of Formula III′, with respect to the weight of the carrier polymer.

19. The diagnostic dental device of claim 1, the carrier polymer comprising units represented by Formula IV′: 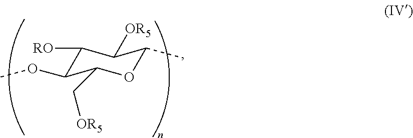
wherein:
each R⁵ is independently selected from —H and —(CH₂)ₚ—OH, and
n is an integer selected from 100, 500, 1000, 2000, 5000, 10000, and 100000, and
p is an integer selected from 1, 2, and 3.

20. The diagnostic dental device of claim 19, the carrier polymer comprising at least 0.1 wt % units of Formula IV′, with respect to the weight of the carrier polymer.

21. The diagnostic dental device of claim 19, the carrier polymer comprising about 0.1 wt % to about 5 wt % units of Formula IV′, with respect to the weight of the carrier polymer.

22. The diagnostic dental device of claim 1, the carrier polymer derived, at least in part, from one or more vinyl dialkylamine monomer represented by Formula (V):
C(R⁶)₂═C(R⁷)—N(R⁸)(R⁹), (V)
wherein:
each R⁶ is independently selected from —H and C₁₋₆ alkyl,
R⁷ is —H or C₁₋₆ alkyl, and
R⁸ and R⁹ are independently selected from a C₁₋₆ alkyl optionally substituted with one or more of —OR¹⁰, —N(R¹⁰)₂, and —OH, —SH, —NH₂, and —NHCO—, or
R⁸ and R⁹, or R⁸ and R¹⁰, are taken together with the atoms to which they are attached to form a 5- or 6-membered heterocycle, the 5- or 6-membered heterocycle optionally substituted with oxo.

23. The diagnostic dental device of claim 1, the carrier polymer comprising units represented by Formula (V′):
—[—C(R⁶)₂—C(R⁷)(N(R⁸)(R⁹))—]—, (V′)
wherein:
each R⁶ is independently selected from —H and C₁₋₆ alkyl,
R⁷ is —H or C₁₋₆ alkyl, and
R⁸ and R⁹ are independently selected from a C₁₋₆ alkyl optionally substituted with one or more of —OR¹⁰, —N(R¹⁰)₂, and —OH, —SH, —NH₂, and —NHCO—, or
R⁸ and R⁹, or R⁸ and R¹⁰, are taken together with the atoms to which they are attached to form a 5- or 6-membered heterocycle, the 5- or 6-membered heterocycle optionally substituted with oxo.

24. The diagnostic dental device of claim 23, the carrier polymer comprising at least 0.1% wt % units of Formula V′, with respect to the weight of the carrier polymer.

25. The diagnostic dental device of claim 24, the carrier polymer comprising about 0.1 wt % to about 5 wt % units of Formula V′, with respect to the weight of the carrier polymer.

26. The diagnostic dental device of claim 1, the carrier polymer comprising units represented by Formula VI′:
—[C(R¹)₂—C(R²)(OH)]—]— (VI′)
wherein:
each R¹ is independently selected from —H and a C₁₋₆ straight or branched alkyl optionally substituted with one or more of —OR⁴, —N(R⁴)₂, —OH, —SH, —NH₂, and —NHCO—, and
R² is —H, or a C₁₋₆ straight or branched alkyl optionally substituted with one or more of —OR⁴, —N(R⁴)₂, —OH, —SH, —NH₂, and —NHCO—.

27. The diagnostic dental device of claim 26, the carrier polymer comprising at least 0.1 wt % units of Formula VI′, with respect to the weight of the carrier polymer.

28. The diagnostic dental device of claim 27, the carrier polymer comprising about 0.1 wt % to about 5 wt % units of Formula VI′, with respect to the weight of the carrier polymer.

29. The diagnostic dental device of claim 1, the carrier polymer comprising a hydrophilic acrylate.

30. The diagnostic dental device of claim 1, wherein the carrier polymer is present in the indicator composition in an amount of about 0.1 wt % to about 5% wt %, with respect to the weight of the indicator composition.

31. The diagnostic dental device of claim 1, wherein Y⁺ is Na⁺.

32. The diagnostic dental device of claim 1, wherein the indicator compound of Formula I is present in the indicator composition in an amount of about 0.001 wt % to about 0.5 wt %, with respect to the weight of the indicator composition.

33. The diagnostic dental device of claim 1, wherein the carrier polymer and the indicator compound of Formula I are present in a weight ratio of about 5:1 to about 1:5.

34. The diagnostic dental device of claim 1, the indicator composition further comprising a supplemental indicator compound represented by Formula VII: 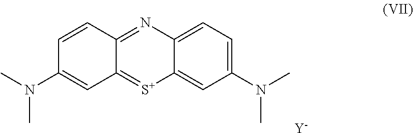
wherein:
Y⁻ is a pharmaceutically acceptable counterion.

35. The diagnostic dental device of claim 34, wherein the supplement indicator compound of Formula VII is present in an amount of about 0.001 wt % to about 0.05 wt %, with respect to the weight of the indicator composition.

36. The diagnostic dental device of claim 34, wherein the indicator compound of Formula I and the supplement indicator compound of Formula VII are present in a molar ratio of about 1:2.5.

37. The diagnostic dental device of claim 34, wherein Y⁻ is selected from Cl⁻, Br⁻, NO₃⁻, HSO₄⁻, SO₄⁽²⁻⁾, and H₂PO₄⁽⁻⁾.

38-48. (canceled)
